(19) United States
(12) Patent Application Publication
(10) Pub. No.: US 2008/0114299 A1
(43) Pub. Date: May 15, 2008

(54) Remote Commander To Be Used With A Drug Delivery Device

(76) Inventors: Camilla Damgaard-Sorensen, Frederiksberg (DK); Kristian Glejbol, Glostrup (DK)

Correspondence Address: NOVO NORDISK, INC.;PATENT DEPARTMENT, 100 COLLEGE ROAD WEST, PRINCETON, NJ, 08540, US

(21) Appl. No.: 11/662,986

(22) PCT Filed: Sep. 20, 2005
(86) PCT No.: PCT/EP05/54676

§ 371 Date: Aug. 13, 2007

(30) Foreign Application Priority Data
Sep. 23, 2004 (DK) PA 200401447

(51) Int. Cl. A61M 5/142

(52) U.S. Cl. 604/131; 604/264

(57) ABSTRACT

The invention relates to a remote commander adapted to be used in combination with a drug delivery device comprising drug expelling means and a delivery receiver The remote commander comprises user input means allowing a user to input data, and a transmitter adapted for wirelessly transmitting data to the delivery receiver for controlling the expelling means. The user input means comprises a dose setting member which is moveable in a first direction to a selected set position representing a set dose of drug to be delivered, as well as moveable in a second direction to selectively adjust the set position and thereby the set dose of drug. The user input means further comprises an actuation member providing that data representing the set dose can be transmitted to the delivery device to thereby expel the set dose of drug.

The invention relates to a remote commander for use with a remotely controllable drug delivery device, the remote commander being adapted to receive input user data and transmit such data to the drug delivery device.

### BACKGROUND OF THE INVENTION

In the disclosure of the present invention reference is mostly made to the treatment of diabetes by injection or infusion of insulin, however, this is only an exemplary use of the present invention. For example, in embodiments of the present invention the device may serve as a motivator or support for lifestyle adjustments, e.g. in the treatment of obesity or cardiovascular deceases.

Managing a chronic disease or ongoing health condition normally requires the patient to follow a regimen. Such a regimen may be very simple such as taking three pills each day or it may be more complicated such as e.g. in diabetes in which the patient in addition to taking a scheduled medication, e.g. one or more oral anti diabetic drugs (OAD) and/or one or more injections with a basal insulin, also will have to follow a regimen in terms of e.g. exercise, meals and meal-related injections of an insulin bolus as well as measuring the blood glucose.

In the latter case, the patients ideally monitor and control the parameters relevant for their decease in clinician assisted self-care treatment programs. In these treatment programs, patients are responsible for performing self-care actions which impact the control parameter, e.g. the blood glucose measurements. Patients are also responsible for measuring the control parameter to determine the success of the self-care actions and the need for further adjustments. The successful implementation of such a treatment program requires a high degree of motivation, training, and understanding on the part of the patients to select and perform the appropriate self-care actions.

To help assist the patient in achieving the set goals, e.g. a certain blood glucose reflecting value, diabetes management systems have been proposed. For example, U.S. Pat. No. 6,379,301 discloses a diabetes management system for predicting a future blood glucose value of a patient and for recommending a corrective action to the patient when the future blood glucose value lies outside of a target range. The system includes a patient-operated apparatus for measuring blood glucose values and for storing data relating to insulin doses administered to the patient. The apparatus predicts the patient's future blood glucose value based upon the patient's current blood glucose value, the fraction of insulin action remaining from the insulin doses, and the patient's insulin sensitivity. The apparatus also determines the corrective action for the patient when the predicted blood glucose value lies outside of a target range, e.g. injecting an amount of insulin for a diabetes type 1 patient. In embodiments, the apparatus may also stores guidelines for diet, exercise, and other therapy parameters. Further, the apparatus may be programmed to prompt a patient for data relating to the therapy parameters and to display recommended guidelines to the patient.

U.S. Pat. No. 6,352,505 discloses a device for management of diabetes and comprising a programmable microprocessor based unit having a display, keyboard, and memory, the keyboard for inputting information concerning carbohydrates ingested by a user, the microprocessor being adapted to determine an amount of insulin to be used by a user based upon the carbohydrates ingested and the display displaying the amount of insulin to be used. This device also enables an individual to factor in other variables besides carbohydrate food content such as exercise.

These kinds of systems may be stand-alone products or they may be incorporated into a drug delivery device or system. For example, a drug delivery system may comprise a drug delivery device, e.g. a body-worn pump, and a remote control for wirelessly controlling the pump, the management system and patient interface being incorporated into the remote control device. Such a system may also comprise a build-in glucose meter or it may be adapted to communicate with a meter or device for continuous blood glucose monitoring.

Although this kind of system provides almost all the means necessary to achieve the “perfect” treatment of a patient suffering from diabetes, it is very complicated to operate typically requiring the patient to operate a menu-based interface, the interface comprising many different menus as well as a large number of keys. Being adapted primarily for “advanced” regimens this kind of management will not be suitable for patients following only a “simple” regimen, just as the known systems generally are not adapted to serve as an instrument for securing that a given regimen is followed.

Managing a chronic disease or ongoing health condition may also include change of regimen in respect of the device used to control the disease. For example, a patient may shift from a regiment based on drug injections using a pen to a regiment based on a remotely controllable drug infusion device, e.g. of the type disclosed in US 2003/0212379 which is hereby incorporated by reference.

### DISCLOSURE OF ASPECTS OF THE INVENTION

Having regard to the above, it is an object of the present invention to provide a device or system which can be useful to assist a patient in following a given self-care regimen which typically will have been agreed upon with the patients attending physician. The device or system should be easy to understand and use to thereby achieve a high degree of compliance with and understanding of the regimen. It is a further object to provide a system which can be adapted to suit different levels of self-care regimens and which can be adapted to the changing needs of a given patient. For example, the system may be adaptable from the situation in which a patient uses a manual pen-based drug injection device, to the situation in which the patient starts to use an “advanced” pump-based drug infusion system. Such a system may comprise a body-worn pump and a remote control for wirelessly controlling the pump.

Addressing the object of the changing needs of the patient, it is a further object of the present invention to provide a remote control unit for a remotely controllable drug infusion device which will provide an easy transition for a patient shifting from a regiment based on drug injections using a pen to a regiment based on a remotely controllable drug infusion device.

In the disclosure of the present invention, embodiments will be described which will address one or more of the above objects or which will address objects apparent from the below disclosure as well as from the description of exemplary embodiments.

Thus, corresponding to a first aspect, an indication device is provided comprising a first display area including at least one of a first indicator representative of a first type of activity, and a second display area comprising at least one of a second indicator representative of a second type of activity, wherein each indicator has a first visual state and a second visual state. The indication device further comprises input means allowing each indicator to be switched from the first to the second visual state (which may also be termed the activated state), and indication controller means for controlling the display areas and the user input means. One or more display areas may comprise at least two indicators. In preferred embodiments the switching of a selected indicator from the first to the second visual state does not influence the visual state of other indicators. Alternatively, previously changed indicators may be changed when a most recent indicator is changed.

The term “indication device” is used to denote that a feature of the present invention is to provide visual information to the user indicative of compliance with a given regimen.

As the indication device provides at least two distinct display areas, it is possible for each area to display information in respect of a specific aspect of a given regiment without the user (which will typically be the patient) having to switch between different display modes. In this way important information will not be overlooked as it constantly will be on display. Indeed, the number of display areas will determine how many different types of regiment aspects can be displayed at any given time. In each display area one or more indicators are displayed, each display area representing an activity to be performed by the patient. For example, in a given display area three indicators may be shown indicating that three tasks have to be dealt with. In case a larger number of indicators has to be displayed than would be suitable for a given display area, an additional indicator may indicate that there are additional tasks to be performed.

To allow the device to indicate that a given activity has been performed, each indicator has a first and a second visual state, which typically will represent a task to be done and a task completed. To control the switching between the two states, the indication device comprises input means allowing the user to control the state of the individual indicators. The input means may be in the form of input keys or the symbols may be automatically switched when a given task is performed, e.g. the device may include a blood glucose meter (BGM) which will switch a signal when the BGM is used, or it may be adapted to wirelessly receive BGM data. To control operation of the device, controller means is incorporated, e.g. electronic circuitry which typically will incorporate one or more micro-processors. The one or more processors may be supplemented by additional devices for support or control functions. For example, a transmitter and receiver may be fully or partly integrated with a processor, or may be provided by individual units. Each of the components making up the processor circuitry may be special purpose or general purpose devices. In preferred embodiments the display areas are formed by one or more display screens, e.g. of the LCD (liquid crystal display) type, allowing indicators in graphical form to be displayed, e.g. icons, or in simpler versions by a number of LED (light emitting diode) devices, e.g. a given indicator may be a LED shifting between two states. In exemplary embodiments the controller means will also allow the attending physician to set up (i.e. program) the device to correspond to a given regimen for a given patient. For example, the physician may decide as to the number of display areas, the type of task assigned to each display area, the number of indicators in each area, and eventually the information or options associated with a given indicator. Further, the controller means may provide a memory which later can be accessed by the physician to control compliance. The regimen ordered by the physician will typically consist of a number of tasks to be performed daily for which reason the device advantageously comprises a timer which resets the device each night such that the patient will wake up to a “fresh” set of tasks to be performed.

To provide further information for the user, the indication device may be provided with a data display area associated with at least one of the indicators or one of the display areas, the data display area being adapted to display data associated with a selected indicator or a selected display area. In this way a second level of information is provided which can be used to display details of a given task or which can be used to provide the user with a number of choices for the given task, i.e. the size or the type of a given meal (see further below). For example, when the user activates a given indicator (e.g. when tapping on it using a touch sensitive display), a message is displayed in the display area associated with the activated indicator, the user input means allowing the user to switch the activated indicator from the first to the second visual state, either by accepting the displayed message or by choosing one of a number of displayed options.

To allow the indication device to be adapted for a diabetic patient with a given regimen, the activities associated with the different display areas may be selected from the following group: intake of food, exercise, drug administration, or measuring a blood parameter such as a blood glucose value. The indicators in the different display areas may be identical or they may be specific for the given area in order to provide for easy visual identification, e.g. icons formed by a LCD. To further provide information to the patient at a glance, the indicators in one or more of the display areas may have a second visual state representing the time at which an individual indicator was switched from its first visual state to its second visual state, e.g. when an exercise was performed or when a meal was taken. Indeed, the device may store the time in its memory without displaying it to the user. The indicators in a display area may have a second visual state representing the amount of a drug associated with that task, e.g. the number of infused or injected insulin units. Each of the indicators may also be associated with a time frame within which a given task is to be performed, e.g. a given indicator may change in configuration if it has not been activated within a given pre-set period of time, e.g. breakfast should be taken between 7.00 and 9.00 in the morning. The indication device may also be provided with an alarm which indicates to the patient that a given (important) task is overdue.

If one of the tasks of the regimen is to take a bolus of insulin in combination with a meal, the size of the dose may be pre-selected in accordance with the type of meal, however, the indication device may be provided with the capacity to receive data representing a patient body blood parameter (e.g. blood glucose) and in response thereto using an algorithm to calculate an amount of a drug (e.g. insulin) representing a recommendation to the user. This type of algorithm is well known in the art, see for example U.S. Pat. No. 6,551,276 which is hereby incorporated by reference. Preferably, the BGM data are automatically transferred to the control means, either by a build-in BGM or directly (e.g. wirelessly) from a separate BGM.

The indication device of the present invention may be provided as a unitary device which is then adaptable for a number of different types of regimens. For example, the device may comprise a single display (e.g. a high resolution LCD) which in accordance with the programming can be used to display one or more display areas and one or more data display areas. If the screen is of the touch-type it may also be used to provide the input means for the patient, however, the device may also be provided with a number of traditional keys, one or more of which may be soft-keys controlled by the display settings.

It should be emphasized that although it is a main feature of the present invention to be able to provide a given patient with an easy-to-use and easy-to-understand representation of the regimen agreed with the doctor, the actual display device which is used to implement the present invention may be of a general type, e.g. a remote commander for a drug delivery system, which may adapted for displaying other types of information, e.g. when setting up or adjusting a drug delivery pump, such that at given times the display do not actually shown the above defined information in the defined areas. However, what is important is that a given device is capable of displaying the set of information as defined and when needed.

However, in order to better suit the individual needs of a given patient and a prescribed regimen, the indication device of the invention may be of a modular configuration allowing the physician to “build” the device by assembling a number of different units.

For example, a master unit may be provided including a main controller and an energy source, the main controller being adapted to control a number of display areas arranged on individual indication units detachably connected to the master unit and controlled by the main controller. According to the needs of the patient one or more such indication units may be attached. A data display as well as user input keys may be provided on the master unit or these may (additionally) be provided on one or more of the indication units.

In an exemplary embodiment the indication device comprises a transmitter coupled to the indication controller means, the transmitter being adapted for wirelessly transmitting data to a receiver. In this way data can be sent to a base unit for storage or transmission to the physician, or the indication device can be used to control a drug delivery device. The indication device may also comprise a receiver coupled to the indication controller means, the receiver being adapted for wirelessly receiving data from a transmitter, e.g. a drug delivery device or a programming unit located with the physician. When used in combination with a drug delivery device switching an activated indicator from the first to the second visual state may generates the transmission of data, e.g. the size of a bolus of insulin to be infused.

The indication device of the invention may also be provided in combination with a drug delivery device comprising a reservoir adapted to contain a fluid drug, expelling means for expelling the fluid drug from the reservoir to a patient, and expelling controller means for controlling the expelling means to deliver a selected amount of drug. In such a system a receiver is coupled to the expelling controller means, the receiver being adapted for wirelessly receiving data from a transmitter arranged in the indication device, such that the received data provides information for controlling the expelling means.

The invention also provides a method for setting up an indication device of the above-disclosed type, the indication device being capable of displaying a plurality of display areas, each display area being capable of displaying at least one indicator of a given type, each indicator having a first visual state and a second visual state, the method comprising the steps of selecting the number of display areas to be displayed, and selecting the number of a given indicator to be displayed in each display area. The method may comprise the further step of for each display area selecting a type of indicator type.

If the indication device in addition comprises at least one data display area adapted to display data associated with a given indicator, the method may comprise the steps additional step of selecting data information to be associated with at least one individual indicator.

In a further aspect of the invention a drug delivery system is provided comprising a drug delivery device and a remote commander, the drug delivery device comprising a reservoir adapted to contain a fluid drug (e.g. pre-filled or user-fillable), an expelling assembly for expelling the fluid drug from the reservoir to a patient, delivery controller means for controlling the expelling means to deliver a selected amount of drug, and a delivery receiver coupled to the delivery controller means, the delivery receiver being adapted for wirelessly receiving data from a commander transmitter arranged in the remote commander, the received data providing information for controlling the expelling means. The remote commander comprises commander controller means, user input means allowing the user to input data, and a commander transmitter coupled to the commander controller means, the commander transmitter being adapted for wirelessly transmitting data to the delivery receiver for controlling the expelling means, the transmitted data representing or being derived from the user input data. In such a system the user input means comprises a dose setting member moveable in a first direction to a selected set position representing a set dose of drug to be delivered and moveable in a second direction to selectively adjust the set position and thereby the set dose of drug, the user input means further comprising a user actuation member, actuation of the user actuation member providing that data representing the set dose is transmitted to the delivery controller means to thereby expel the set dose of drug.

The present invention also provides a remote commander as defined above adapted to be used in combination with a drug delivery device as defined above.

Is this way the primary user-interface of the remote commander (i.e. the structures responsible for selecting and actuating a drug bolus of a given size) will resemble the user-interface provided on a most drug injection devices of the pen-type, this allowing the user to feel familiar with the remote commander when shifting from a pen-based to a pump-based drug regimen. To further imitate the configuration of a pen, the remote commander may comprise a generally cylindrical portion defining an axis, the dose setting member being in the form of a ring or knob arranged on the generally cylindrical portion and being rotatable corresponding to the axis, the user actuation member advantageously being arranged at the end of the generally cylindrical portion. To yet further imitate the configuration of a pen, the user actuation member may be moved from an initial position to an actuatable position when a dose is set.

The selected dose may be shown directly by indicia on the dose setting member, however, the remote commander advantageously comprises a display adapted to display a value corresponding to the selected set dose. For a more advanced type of system, the remote commander may comprise means for inputting data representing a patient body parameter (e.g. blood glucose) to the commander controller means, wherein the commander controller means is responsive to the input data for calculating a dose recommendation (e.g. a bolus dose of insulin), the dose recommendation being transformable to a set dose by either adjusting it using the dose setting member or by actuating the user actuation member. The means for inputting data may for example be keys allowing the user to manually input data, a build-in glucose meter, or a wireless receiver allowing the remote commander to receive data from a separate unit, e.g. a blood glucose meter, a PDA or a computer.

In an exemplary embodiment the drug delivery device further comprises a delivery transmitter coupled to the delivery controller means, the delivery transmitter being adapted for wirelessly transmitting data to a commander receiver, and the remote commander further comprises a commander receiver coupled to the commander controller means, the commander receiver being adapted for wirelessly receiving data from the delivery transmitter. By this arrangement the drug delivery device can be adapted to transmit data to the remote commander confirming that a given set dose has been received from the remote commander and/or that the expelling means has been controlled to expel an amount of drug corresponding to a set and transmitted dose, this allowing the user to feel secure when instructions or data are sent to a drug delivery device concealed by clothing.

The remote commander may incorporate features of the above-described indication device, e.g. the remote commander may comprise a display which serves as an indication device or which have a mode allowing it to serve as an indication device. The commander and delivery controller means may be in the form electronic circuitry typically incorporating one or more micro-processors.

In a further aspect of the invention a method for operating a drug delivery system is provided, the system comprising a drug delivery device and a remote commander, the drug delivery device comprising a delivery receiver and a drug dispenser, the remote commander comprising a commander transmitter, a dose setting member and a user actuation member. The method comprises the steps of moving the dose setting member in a first direction to a selected set position representing a set dose of drug to be delivered, and actuate the user actuation member, whereby actuation of the user actuation member provides that data representing the set dose is transmitted to the delivery receiver to effectuate that the set dose of drug is dispensed. Prior to actuating the user actuation member, the dose setting member may be moved in a second direction to selectively adjust the set position and thereby the set dose of drug. The dose setting member may be moved in a given direction by rotating the dose setting member in the given direction.

As used herein, the term “drug” is meant to encompass any drug-containing flowable medicine capable of being passed through a delivery means such as a hollow needle in a controlled manner, such as a liquid, solution, gel or fine suspension. Representative drugs include pharmaceuticals such as peptides, proteins, and hormones, biologically derived or active agents, hormonal and gene based agents, nutritional formulas and other substances in both solid (dispensed) or liquid form. In the description of the exemplary embodiments reference will be made to the use of insulin. Correspondingly, the term “subcutaneous” infusion is meant to encompass any method of transcutaneous delivery to a subject. Further, the term needle (when not otherwise specified) defines a piercing member adapted to penetrate the skin of a subject.

### BRIEF DESCRIPTION OF THE DRAWINGS

In the following the invention will be further described with references to the drawings, wherein

FIG. 1 shows an indication device having a unitary configuration,

FIG. 2 shows an indication device having a modular configuration,

FIG. 3 shows in a disassembled state an indication device having a modular configuration,

FIG. 4 shows the device of FIG. 3 in an assembled state,

FIG. 5 shows icons representing different tasks,

FIGS. 6A and 6B show the use of a display unit of an indication device,

FIG. 7 shows an example of an indicator in its first and second state,

FIGS. 8A-8C show the use of a further display unit of an indication device,

FIG. 9 shows an embodiment of a remote commander,

FIG. 10 shows an embodiment of a further remote commander, and

FIG. 11 shows a schematic representation of a drug delivery system.

In the figures like structures are identified by like reference numerals.

### DESCRIPTION OF EXEMPLARY EMBODIMENTS

When in the following terms as “upper” and “lower”, “right” and “left”, “horizontal” and “vertical” or similar relative expressions are used, these only refer to the appended figures and not to an actual situation of use. The shown figures are schematic representations for which reason the configuration of the different structures as well as there relative dimensions are intended to serve illustrative purposes only.

FIG. 1 shows an indication device having a generally unitary configuration such that adaptation and the configuration of the device to suit individual patient needs primarily will be based on programming. The indication device will be described focusing primarily on the directly user-oriented features. More specifically, the display device comprises a housing 110, a LCD display 120, a navigation key 170 and two input keys 181, 182. In the shown embodiment the display has been set up by the attending physician (in the following also named doctor) to show three display areas 130, 140, 150 each comprising three indicators 141, 151, 161, the indicators in each area being initially identical and representing a type of activity or task to be performed by the patient. However, a different number of areas can be chosen just as the size and form of the areas and the number or indicators in each area may be different. Each indicator has a first visual state indicating that a task is not yet performed and a second visual state indicating that the task has been performed (see below). The display further comprises a data display area 163 which can display data associated with a selected indicator. The data display comprises two soft-key areas 161, 162 which will indicate the actual function of the two input keys 181, 182, as well as a main area. When the data display area is activated it may enlarge to thereby cover some of the display areas, the data display area returning to its normal size when the task has been completed. The navigation key can control a cursor up and down and right and left. In an initial state the navigation key will control a cursor 121 surrounding an indicator. In the shown embodiment the indicators are programmed to be activated in sequence from left to right, thus the cursor will only be able to move the cursor up or down to point at the first indicator in a given display area which is in its first state. When the cursor is at a selected task (i.e. a selected indicator), one of the soft-keys will indicate that the task can be “activated” when the corresponding key is pressed. When so activated the data main area will disclose information associated with the activated task. The information may explain the contents of the task or provide a number of options which can then be selected by use of the navigation key. When the information has been dealt with the two soft-key areas will allow the patient to accept or dismiss the task. In case it is accepted the corresponding indicator will be switched to its second visual state indicating that the task has been performed. In this way the patient will have to continue until all tasks agreed with the doctor have been performed. The indication device further comprises a data inlet and outlet means which may either be provided by electrical connectors or by a wireless receiver and transmitter, e.g. based on RF or IR, allowing the device to be programmed and stored data to be downloaded, e.g. to a computer when visiting the doctor which can then use the data to evaluate whether the patient is in compliance with the agreed regimen. In addition to the first and second states an indicator may have further states, e.g. an intermediate state to indicate that a given task is “overdue” according to a given timeframe. Such an intermediate state may be a different static configuration of the indicator or “dynamic” state such as flashing. In addition, an alarm, e.g. sound or vibration may be associated with such a state.

In an alternative embodiment the display is touch-sensitive, e.g. a touch-sensitive LCD which would allow the patient to activate the tasks, navigate in the data main area and accept the tasks merely by touching the relevant symbol or information on the display.

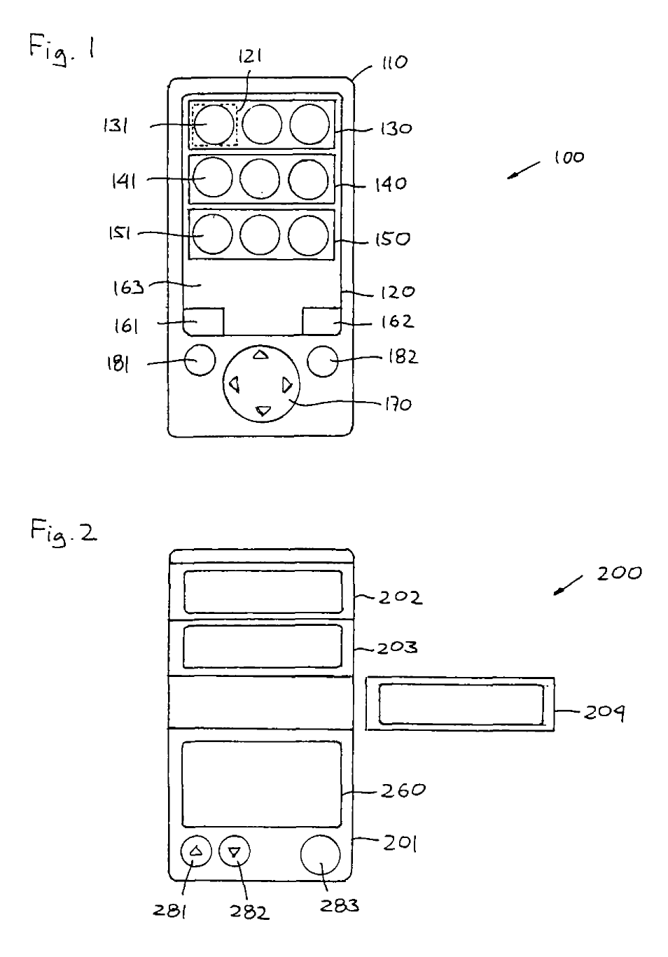

Before turning to a concrete set of tasks, a second embodiment of a display device will be described. More specifically, FIG. 2 shows an indication device 200 having a modular configuration allowing the device to be assembled from a number of different modules to suit the individual patient needs, however, additional settings may still be provided based on programming. The device comprises a main unit 201 having a data display 260 and corresponding keys 281, 282, 283 as well as three slots allowing e.g. up to three display area modules 201, 202, 203, each having a display area, to be mounted and which in this embodiment comprise touch-sensitive LCDs. When not used a dummy may be placed in a given slot. When the patient has activated a task the patient may navigate (if possible) between options in the data area and/or accept the task by pressing the OK key.

Although the above two embodiments provide basically the same functionality, the patient may in the latter case feel that the device has been “build” specifically for him or her and in this way may feel more motivated and more inclined to use the device and follow and perform the tasks set up together with the doctor.

For a specific patient with e.g. diabetes type 1 the following relatively simple set of tasks may be set up: Three meals, three time physical exercise, and two takings of one or more drugs. One or more of these activities or tasks may be associated with an alarm to remind the patient. In this situation the patient will receive an indication device comprising three display areas, the first comprising three icons for a meal, the second three icons for physical exercise and the third two icons for the drugs (for examples of icons see FIG. 5). When activating a given task the data display area will prompt for information. For example, if a meal task is activated a number of items may show up and the patient may “compose” the meal using the options given by the doctor. When the meal has been composed it may be accepted and the task will be indicated as completed. At the same time the data representing the meal is stored (e.g. the time and, if relevant, the composition) for later retrieval by the doctor. When choosing from a menu, the device may be set up to analyse whether the composition of the meal is appropriate and may guide the patient to properly compose a meal. Correspondingly, when an exercise task is activated a number of items may show up and the patient may choose in accordance with the options given by the doctor. When the choice (if any) has been made it is accepted and the task will be indicated as completed. At the same time the data representing the exercise is stored.

For another patient with e.g. diabetes type 2 the tasks will typically be more comprehensive and it may be relevant to use a more advanced implementation of the principles of the present invention, namely a controller for a drug (insulin) delivery system comprising an electronically controlled body-worn drug infusion pump providing infusion of insulin at corresponding to a basal rate, in combination with a remote commander (RC) therefore and a BGM. The present invention may then be incorporated in the remote commander comprising a suitable display and input means. The remote commander should also be adapted to communicate with the BGM to effectively secure that blood glucose (BG) measurements are transferred to the system. Using such a system, the following more elaborate set of tasks may be set up: Three meals, three bolus injections of insulin, three time physical exercise, and 6 times making a BG measurement.

A given day may start with the patient completing a BG measurement using a BGM as the first task. The BGM may be incorporated in the RC or the data representing the measurement may be transmitted to the RC, e.g. using a wireless means of transmission. The next task would be to take the first meal which may be effectuated as described above, however, the completed meal task may prompt the patient to the insulin task. In an advanced implementation the RC would comprise software and circuitry which based e.g. on one or more previous BG measurements, the actually planed meal as well as other information representing the patient would recommend a bolus of a given calculated size which then could be accepted by the patient which would then have completed the bolus task. The physical exercise tasks may be effectuated as described above.

Although the regimens for the above two examples are very different, the user-interface being based on a tasks-to-be-completed form of communication basically remains the same, this allowing the patient to familiarize with the system at an early stage, the system being adaptable to the patients needs, either as the doctor refines the regimen for a given decease or a given state of decease, or more the decease evolves as may be the case with diabetes. That the system is able to “grove” with the patient does not necessarily mean that it will be the same indication device that the patient will be using, but only that each indication device will assist the patient by using the same task-oriented easy-to-use and easy-to-understand representation of the regimen. For example, in very simple implementation the device may be in the form of e.g. a key fob which merely displays the tasks to be completed, e.g. to take a given medication twice a day and exercise three times, whereas the above-described RC would represent an advanced implementation on the very same principles of the present invention.

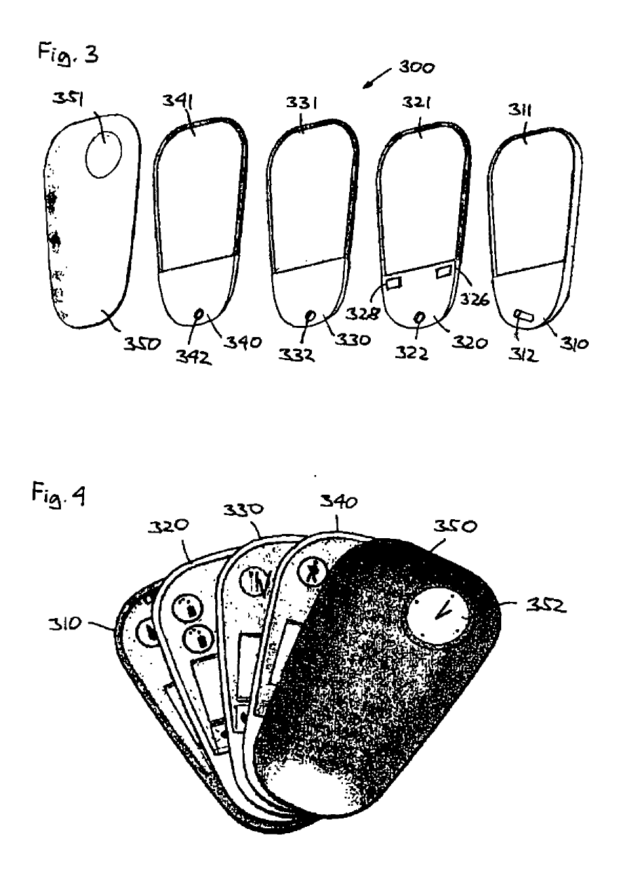

With reference to FIGS. 3-8 a further embodiment of the invention will be described, the indication device being of a RC 300 of modular design comprising a main unit 310, three additional display units 320, 330, 340 and a cover unit 350. The main unit comprises a touch-screen LCD 311, a BGM with an associated port (not shown) for receiving a BG strip, a RF transmitter for transmitting data, a RF receiver for receiving data, a processor for controlling the device and providing the necessary computing of data, and an energy source. The main unit further comprises a connection pin 312 allowing one or more display units to be attached to and controlled from the main unit. Each display unit comprises a touch-screen LCD 321, 331, 341, connection openings 322, 332, 342 for the pin, and may also comprise additional input keys 328, 326. The cover unit primarily serves to protect the uppermost display unit, however, in the shown embodiment it is provided with a small LCD 351 which may be used as a clock 352 or as an alert for the most urgent task to be performed. As will appear, the four displays are arranged in a stack for which reason the units can rotate around the pin and thus relative to each other, e.g. as playing cards, this as shown in FIG. 4. This arrangement provides four relatively large displays in a single compact device.

In the shown embodiment are the four displays dedicated to different types of tasks, and in order from the top: Exercise, meals, bolus infusion of insulin and BG measurement. The respective indicators 335, 345, 325, 315 for the four displays are shown in FIG. 5 in their first state, i.e. corresponding to an active to-be-performed task.

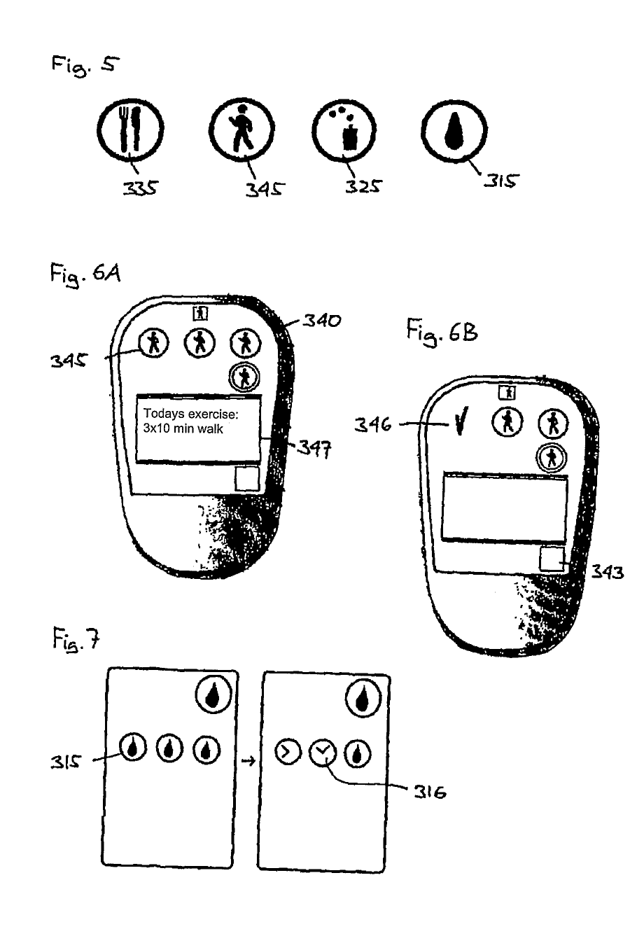

When for example it is time for exercise the patient chooses the exercise unit 340 and taps on one of the icons 345 (indicators) which are located in an upper display area of the LCD, the icon representing a to-be-performed exercise task. When the task is activated (see FIG. 6A) an associated text is displayed in the data display area 347 of the LCD, the text describing the exercise to be performed, this as agreed with the doctor. When accepted by tapping on the OK area 343 of the display, the activated icon is switched to state 346 indicating a “done” task state (see FIG. 6B), where after the patient is supposed to perform the task. Instead of the “V” sign for a done task, the second state of a given indicator may symbolise the actual time when the task was completed as shown in FIG. 7 where the icon 315 for a BG measurement has been switched to clock icon 316.

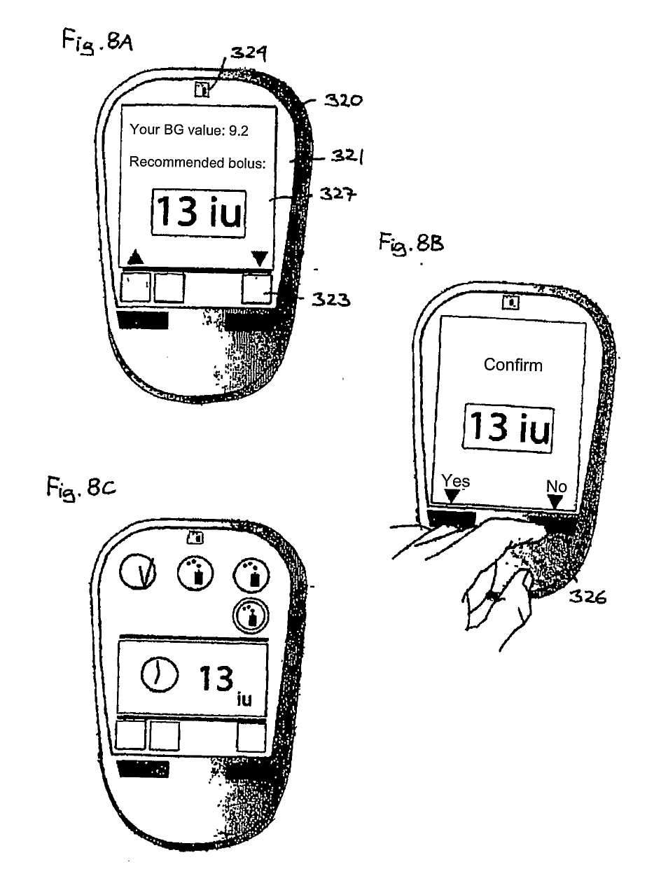

When it is time for a bolus infusion the patient chooses the bolus unit and taps on one of the icons (see FIG. 4) to activate the bolus menu system. As appears in FIG. 8A the data display area 327 enlarges to cover most of the LCD 321, a small icon 324 at the top of the display indicating the actual unit. The display informs the user of the selected meal (a bolus will normally be taken in combination with a meal for which reason the meal unit has been used to indicate this before turning to the bolus task), the most recent BG measurement and the recommended size of the bolus, here 13 IU. The patient may adjust the dose or go to the next menu by tapping on the relevant display keys 323. The next menu will show the selected dose which can be rejected (which will cause the menu to be left without the task being completed) or accepted by pressing the relevant key 326, see FIG. 8B. This will result in a corresponding command being sent to the patient's insulin infusion pump and when the remote commander has received the confirmation signal from the infusion pump the display will indicate the task as completed and the size of the bolus at the time of delivery will be displayed and stored, see FIG. 8C.

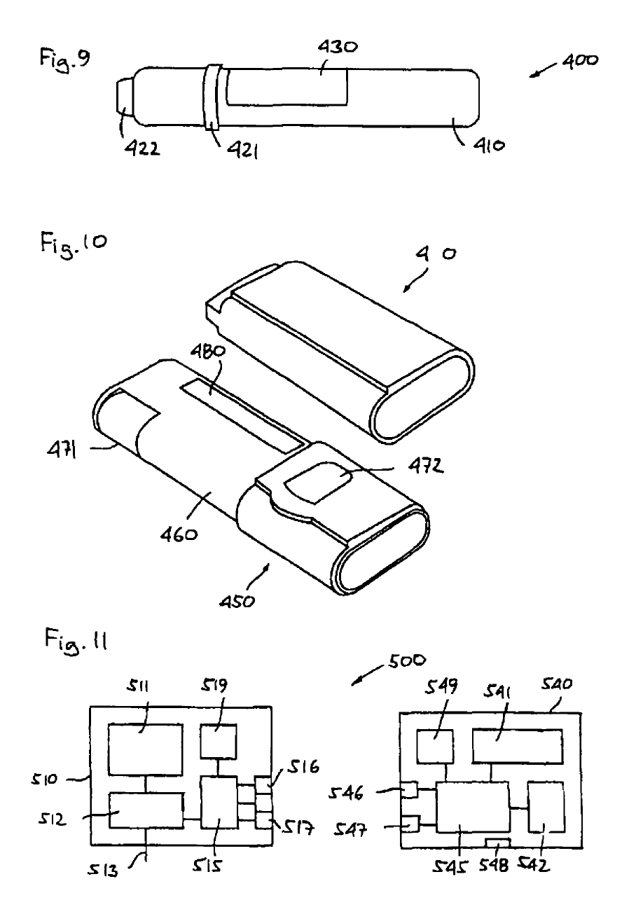

FIG. 9 shows a further remote commander (RC) unit embodying a further aspect of the invention, the RC being adapted to control a drug infusion pump, e.g. as shown in FIG. 11. The remote commander 400 comprises a housing 410 having a generally cylindrical outer shape defining an axis and resembling a pen and thus also a pen-shaped drug injection device. To serve as a control unit for a pump the remote commander comprises commander controller means, user input means allowing the user to input data, and a commander transmitter coupled to the commander controller means, the commander transmitter being adapted for wirelessly transmitting data to a pump receiver for controlling the expelling means of the pump, the transmitted data representing or being derived from the user input data. The user input means comprises a dose setting member 421 in the form of a ring arranged on the generally cylindrical body and being rotatable corresponding to the axis in a first direction to a selected set position representing a set dose of drug to be delivered and rotatable in the reverse direction to selectively adjust the set position and thereby the set dose of drug. The user input means further comprises a user actuation member 422 arranged at the end of the generally cylindrical housing as well as a display 430 adapted to display a value corresponding to the selected set dose. When a dose has been set, actuation of the actuation member by the user provides that data representing the set dose is transmitted to the delivery controller means to thereby expel the set dose of drug. Preferably the remote commander is adapted to receive a confirmation signal from the pump.

As shown above, the well-known user-interface from a pen-formed drug injection device can be applied to a remote commander having the same generally cylindrical configuration, however, the user-interface for a RC in accordance with aspects of the present invention can also be implemented in a RC having a different outer shape. Correspondingly, FIG. 10 shows an embodiment 450 of a RC having a generally box-shaped body, such a configuration allowing e.g. a larger display 480 to be used. In the shown embodiment is the dose setting member a cylindrical member 471 arranged at an edge portion of the body, the actuation member 472 being arranged on top of the upper surface. In the shown embodiment the RC is further provided with a detachable cap 490 to protect the display when the RC is not in use.

The displays of the RCs 450, 480 may incorporate features of the above-described indication device, e.g. the displays may also serve as an indication device or the RCs may have a mode allowing it to serve as an indication device. Indeed, a relatively small display would allow fewer features to be implemented.

FIG. 11 shows a schematic representation of a further embodiment of the invention in the form of a drug delivery system 500 (here: infusion system) comprising a pump unit 510 in combination with a remote control unit 540 (i.e. a remote commander), the combination providing an operative drug delivery system. The RC may correspond to the previously described embodiments.

The pump unit comprises a drug reservoir 511 and a pump assembly 512 comprising an outlet 513 and adapted for infusing a drug into a body of a user in accordance with instructions (i.e. a command) received from a local processor 515. The pump assembly may be of the metering type, i.e. the amount of drug infused corresponds to the controlling signals received from the local processor or the infusion unit may be provided with detecting means for determining the amount of drug actually infused (not shown). The local processor is associated with a local receiver 516 cooperating with the local processor means for receiving control commands from the control unit. The pump unit further comprises a local transmitter 517 cooperating with the local processor means for transmitting data information to a control receiving means 547 provided in the control unit 540. An energy source 519 is provided in the form of a battery.

The control unit comprises a control processor 545 associated with a transmitter 546 for wireless transmitting control commands to the local processor via the local receiver. The control unit further comprises a display 541 associated with the control processor, e.g. corresponding to the display 120 in the first embodiment, input means 548 (e.g. keys or a touch sensitive display) allowing a user to input commands or data to the processor, and memory circuitry 542 allowing transmitted and/or received commands/data to be stored and recalled. An energy source 549 is provided in the form of a battery.

In the above description of the exemplary embodiments, the different structures providing mechanical, electrical and fluid contact and communication between the different components just as the means providing the described functionality for the different components (i.e. pump, reservoir, energy source, memory, control, display etc.) have been described to a degree to which the concept of the present invention will be apparent to the skilled reader. The detailed construction and specification for the different components are considered the object of a normal design procedure performed by the skilled person along the lines set out in the present specification. For example, further details in respect of drug delivery systems, remote commanders and dose calculation means are considered well known in the art and are readily available in order to exercise the principles of the present invention as described above. For example, in respect of drug delivery systems, remote commanders and dose calculation means detailed information is disclosed in U.S. Pat. Nos. 6,551,276 and 6,571,128 which are hereby incorporated by reference.

1. A drug delivery system (500) comprising a drug delivery device (510) and a remote commander (540, 400, 450), the drug delivery device comprising:
a reservoir (511) adapted to contain a fluid drug,
an expelling assembly (512) for expelling the fluid drug from the reservoir to a patient,
delivery controller means (515) for controlling the expelling assembly to deliver a selected amount of drug,
a delivery receiver (516) coupled to the delivery controller means, the delivery receiver being adapted for wirelessly receiving data from a commander transmitter (546) arranged in the remote commander, the received data providing information for controlling the expelling means,
the remote commander comprising:
commander controller means (545),
user input means (548) allowing the user to input user data,
a commander transmitter (546) coupled to the commander controller means, the commander transmitter being adapted for wirelessly transmitting data to the delivery receiver for controlling the expelling means, the transmitted data representing or being derived from the user input data,
wherein the user input means comprises a dose setting member (421, 471) moveable in a first direction to a selected set position representing a set dose of drug to be delivered and moveable in a second direction to selectively adjust the set position and thereby the set dose of drug, the user input means further comprising a user actuation member (422, 472), actuation of the user actuation member providing that data representing the set dose is transmitted to the delivery controller means to thereby expel the set dose of drug.

2. A drug delivery system as in claim 1, wherein the remote commander comprises a generally cylindrical portion (410) defining an axis, the dose setting member being in the form of a ring (421) or knob arranged on the generally cylindrical portion and being rotatable corresponding to the axis.

3. A drug delivery system as in claim 2, wherein the user actuation member (422) is arranged at the end of the generally cylindrical portion.

4. A drug delivery system as in claim 1, wherein the generally cylindrical portion has a free end portion at which the user actuation member is arranged.

5. A drug delivery system as in claim 1, wherein the user actuation member is moved from an initial position to an actuatable position when a dose is set.

6. A drug delivery system as in claim 1, the remote commander comprising a display (430, 480) adapted to display a value corresponding to the selected set dose.

7. A drug delivery system as in claim 1, the remote commander comprising means for inputting data representing a patient body parameter to the commander controller means.

8. An drug delivery system as in claim 7, the commander controller means being responsive to the input data for calculating a dose recommendation, the dose recommendation being transformable to a set dose by either adjusting it using the dose setting member or by actuating the user actuation member.

9. A drug delivery system as in claim 8, wherein the input data represents a patient blood parameter.

10. A drug delivery system as in claim 1, wherein the drug delivery device further comprises:
a delivery transmitter (517) coupled to the delivery controller means, the delivery transmitter being adapted for wirelessly transmitting data to a commander receiver, the remote commander further comprising:
a commander receiver (547) coupled to the commander controller means, the commander receiver being adapted for wirelessly receiving data from the delivery transmitter,
wherein the drug delivery device is adapted to transmit data to the remote commander confirming that a given set dose has been received from the remote commander and/or that the expelling means has been controlled to expel an amount of drug corresponding to a set and transmitted dose.

11. A drug delivery system as in claim 1, wherein the remote commander further comprises:
a first display area comprising at least two of a first indicator representative of a first type of activity,
a second display area comprising at least two of a second indicator representative of a second type of activity,
each indicator having a first visual state and a second visual state,
input means allowing each indicator to be selected and to be switched from the first to the second visual state, and
indication controller means for controlling the display areas and the user input means.

12. A remote commander (540, 400, 450) adapted to be used in combination with a drug delivery device, the drug delivery device comprising:
a reservoir adapted to contain a fluid drug,
expelling means/assembly for expelling the fluid drug from the reservoir to a patient,
delivery controller means for controlling the expelling means to deliver a selected amount of drug,
a delivery receiver coupled to the delivery controller means, the delivery receiver being adapted for wirelessly receiving data from a commander transmitter arranged in the remote commander, the received data providing information for controlling the expelling means,
the remote commander comprising:
commander controller means,
user input means allowing the user to input data,
a commander transmitter coupled to the commander controller means, the commander transmitter being adapted for wirelessly transmitting data to the delivery receiver for controlling the expelling means, the transmitted data representing or being derived from the user input data,
wherein the user input means comprises a dose setting member moveable in a first direction to a selected set position representing a set dose of drug to be delivered and moveable in a second direction to selectively adjust the set position and thereby the set dose of drug, the user input means further comprising a user actuation member, actuation of the user actuation member providing that data representing the set dose is transmitted to the delivery controller means to thereby expel the set dose of drug.

13. A method of operating a drug delivery system comprising a drug delivery device and a remote commander, the drug delivery device comprising a delivery receiver and a drug dispenser, the remote commander comprising a commander transmitter, a dose setting member and a user actuation member, the method comprising the steps of:
moving the dose setting member in a first direction to a selected set position representing a set dose of drug to be delivered,
actuate the user actuation member, whereby actuation of the user actuation member provides that data representing the set dose is transmitted to the delivery receiver to effectuate that the set dose of drug is dispensed.

14. A method as in claim 13, comprising the additional step of:
prior to actuating the user actuation member, moving the dose setting member in a second direction to selectively adjust the set position and thereby the set dose of drug.

15. A method as in claim 13, wherein moving the dose setting member in a given direction includes rotating the dose setting member in the given direction.
